(19) United States
(12) Patent Application Publication
(10) Pub. No.: US 2024/0132933 A1
(43) Pub. Date: Apr. 25, 2024

(54) IMPROVED ANTI-BIOFILM ASSAY METHODS

(71) Applicant: Adaptive Phage Therapeutics, Inc., Gaithersburg, MD (US)

(72) Inventor: Waqas Nasir CHAUDHRY, Gaithersburg, MD (US)

(21) Appl. No.: 18/533,920

(22) Filed: Dec. 8, 2023

Related U.S. Application Data
(63) Continuation of application No. PCT/US2022/032525, filed on Jun. 7, 2022.
(60) Provisional application No. 63/208,173, filed on Jun. 8, 2021.

(51) Int. Cl. C12Q 1/18

(52) U.S. Cl. CPC C12Q 1/18

(57) ABSTRACT

A method for determining the anti-biofilm activity of an active agent on a bacterial biofilm. The method comprises culturing a bacterial colony in a culture medium in contact with at least one bead under conditions to form at least one biofilm coated bead. The at least one biofilm coated bead is then separated from planktonic bacteria to provide at least one washed biofilm coated bead substantially free of planktonic bacteria. The washed biofilm coated bead is then aseptically transferred to a vessel such as a well of a well plate. A growth medium, a metabolic dye and a known amount of the active agent of interest are then added to the or each vessel containing a washed biofilm coated bead. The growth medium, metabolic dye, active agent of interest and biofilm coated bead are then incubated in the or each vessel. Any change in color intensity of the metabolic dye in the or each vessel is measured and the activity of the active agent against the bacterial biofilm is determined based on the color intensity in the or each vessel.

### TECHNICAL FIELD

The present disclosure relates to generally to antibacterial assays and more particularly to assays to determine the efficacy of active agents to biofilms.

### BACKGROUND

Biofilms are complex three-dimensional bacterial communities adhering to a surface and encased in a hydrated extracellular matrix. The formation of biofilms on biological and inanimate surfaces presents significant medical problems and biofilms are often associated with many pathogenic forms of human diseases and plant infections.

Biofilm related infections are typically treated with antibiotics or antibiotic combinations that are optimized to treat infections caused by planktonic bacteria. Unfortunately, bacteria in the biofilm mode of growth are highly resistant to treatment with antibiotics. Therefore, despite the fact that antibiotics achieve therapeutic concentrations in the blood, biofilm infections often persist until the infected surface is removed.

Antibiotic and phage treatment are two first line treatments for biofilm related infections. However, there is a lack of correlation between conventional biofilm susceptibility test results and therapeutic success in chronic infections. Therefore, several in vitro models to evaluate antimicrobial activity on biofilms have been developed. For example, the minimum inhibitory concentration of an antibiotic to biofilm is commonly determined in vitro by one of two widely used methods, namely the tetrazolium dye method for determination of minimum biofilm inhibitory concentration (MBIC) of an antibiotic and the MBEC Assay™ (formerly Calgary biofilm device) method.

Briefly, in the tetrazolium dye method cultures of the bacterial strain are incubated in well plates and the presence of biofilm in the wells is determined by metabolic dye-reduction assay method using MTT (3-(4,5-dimethylthiazol-2-yl)-2,5-diphenyltetrazolium bromide). In this assay, the live cells reduce the dye leading to color formation which can be read at 570 nm and the intensity of color can be correlated to number of live cells. For biofilm inhibitory studies, the wells are challenged with various concentrations of antibiotic drugs, the contents of the wells are aspirated out and the biofilm adhered to the wells is quantified using the MTT assay as described above. The MBIC is defined as the minimum concentration of drug showing no color development (Nair et al., 2016).

In the MBEC Assay™ biofilms are established on 96 pegs extending from a plastic lid and antimicrobial efficacy testing is carried out by inserting the 96 biofilm coated pegs into a 96 well plate. The assay design allows for the simultaneous testing of multiple biocides at multiple concentrations with replicate samples.

There are two methods widely reported in the literature which can be used to screen bacteriophages (‘phages’) for anti-biofilm activity in vitro. The optical density based assay is assays used to investigate the biofilm formation and phage treatments. Briefly, biofilms are grown in microtitre plates and phage is added to the washed wells of the microtiter plate and incubated to allow the phage to stop/kill biofilm bacterial population. Once the treatment time ends, the plates are stained with Crystal Violet (CV) which binds with the biofilm mass on the walls of the wells. The CV dye amount is measured by measuring optical density at 590 nm (Merrit et al., 2005). In the live cell count method, biofilm cultures are treated with the phage and after treatment the cells are rendered planktonic by scraping the biofilm from walls of the well with the wooden applicator and later forced through a syringe needle to homogenize the cultures. The densities of viable bacteria and of phage are estimated by serial dilution and plating (Chaudhry et al., 2017).

Synergistic phage-antibiotic combinations have also been used to treat biofilm related infections and Crystal Violet phage-antibiotic biofilm synergy testing assays (Comeau et al., 2007) and live cell count assays (Chaudhry et al., 2017) have also been used to screen phage-antibiotic synergy for anti-biofilm activity in-vitro.

Unfortunately, many conventional assay methods are time consuming with some taking 3-5 days to produce results. Furthermore, many conventional assay methods do not provide results in real time and/or do not produce robust and reproducible results. The applicant is also not aware of any current commercial diagnostic test for determining minimum biofilm inhibitory concentration (MBIC) for bacterial biofilms.

There is thus a need to provide in-vitro methods for determining anti-biofilm activity of active agents such as antibiotics, phage and antibiotic-phage combinations that overcome one or more of the problems associated with conventional methods.

### SUMMARY

According to a first aspect of the present disclosure, provided herein is a method for determining the anti-biofilm activity of an active agent on a bacterial biofilm, the method comprising:
culturing a bacterial colony in a culture medium in contact with at least one bead under conditions to form at least one biofilm coated bead;
separating the at least one biofilm coated bead from planktonic bacteria to provide at least one washed biofilm coated bead substantially free of planktonic bacteria;
transferring a washed biofilm coated bead to a vessel;
adding a growth medium, a biofilm dye and a known amount of an active agent of interest to the vessel containing a washed biofilm coated bead;
incubating the growth medium, biofilm dye, active agent of interest and biofilm coated bead in the or each vessel;
measuring any change in color intensity of the metabolic dye in the or each vessel; and
determining the activity of the active agent on the bacterial biofilm based on the color intensity in the or each well.

In some embodiments, the active agent is an antibiotic.

In some embodiments, the active agent is a bacteriophage.

In some embodiments, the active agent comprises an antibiotic and a bacteriophage or more than one bacteriophage.

In some embodiments, the method comprised:
adding a first known amount of the active agent of interest to a first vessel containing a washed biofilm coated bead;
adding at least one other known concentration of the active agent of interest to at least one other vessel containing a washed biofilm coated bead; and
determining the activity of the active agent on the bacterial biofilm based on differences between the intensity of the color in the first well and the other vessel(s).

In some embodiments, the biofilm dye is a metabolic dye such as 3-(4,5-dimethylthiazol-2-yl)-2,5-diphenyltetrazolium bromide (MTT).

In some embodiments, the growth medium is tryptic soy broth (TSB).

In some embodiments, the bacterial colony is a single bacterial colony.

According to a second aspect of the present disclosure, provided herein is a method for determining the minimum biofilm inhibitory concentration of an active agent, the method comprising:
culturing a bacterial colony in a culture medium in contact with at least one bead under conditions to form at least one biofilm coated bead;
separating the at least one biofilm coated bead from planktonic bacteria to provide at least one washed biofilm coated bead substantially free of planktonic bacteria;
transferring a washed biofilm coated bead to a well of a multi-well plate;
adding a growth medium, a biofilm dye and a first known amount of an active agent of interest to a first well containing a washed biofilm coated bead;
adding a growth medium, a biofilm dye and a second known amount of the active agent of interest to a second well containing a washed biofilm coated bead; and
incubating the growth medium, biofilm dye, active agent of interest and biofilm coated bead in the first and second wells;
measuring any change in color intensity of the metabolic dye in the first and second wells; and
determining the minimum biofilm inhibitory concentration of the active agent on the bacterial biofilm based on differences between the intensity of the color in the first well and the second well.

### BRIEF DESCRIPTION OF DRAWINGS

Embodiments of the present disclosure will be discussed with reference to the accompanying drawings wherein:

FIG. 1 is a schematic diagram illustrating an embodiment of the present disclosure.

FIG. 2 shows the layout of the 96 well plate with beads suitable for use in an MBIC assay in accordance with embodiments of the present disclosure.

FIG. 3 shows the metabolic activities of planktonic (top two panels) and biofilm (Bottom two panels) bacteria in the presence of phage (t) and absence of phage (control).

FIG. 4 shows the metabolic activities of planktonic and biofilm bacteria in the presence of phage, antibiotic or their combination.

### DESCRIPTION OF EMBODIMENTS

Details of terms used herein are given below for the purpose of guiding those of ordinary skill in the art in the practice of the present disclosure. The terminology in this disclosure is understood to be useful for the purpose of providing a better description of particular embodiments and should not be considered limiting.

Unless otherwise explained, all technical and scientific terms used herein have the same meaning as commonly understood by one of ordinary skill in the art to which this disclosure belongs.

In the context of the present disclosure, the terms “about” and “approximately” are used in combination with an amount, number, or value, then that combination describes the recited amount, number, or value alone as well as the amount, number, or value plus or minus 10% of that amount, number, or value. By way of example, the phrases “about 40%” and “approximately 40%” disclose both “40%” and “from 36% to 44%, inclusive”.

In the context of the present disclosure, “effective amount”, and related terms, means an amount needed to induce a desired response.

In the context of the present disclosure, “phage”, and related terms, means a bacteriophage which is a bacterial virus.

The singular terms “a,” “an,” and “the” include plural referents unless context clearly indicates otherwise. The term “comprises” means “includes.” Therefore, comprising “A” or “B” refers to including A, including B, or including both A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described herein. In case of conflict, the present specification, including explanations of terms, will control. In addition, the materials, methods, and examples are illustrative only and not intended to be limiting.

Disclosed herein is a method for determining the anti-biofilm activity of an active agent on a bacterial biofilm. The method comprises culturing a bacterial colony in a culture medium in contact with at least one bead under conditions to form at least one biofilm coated bead. The at least one biofilm coated bead is then separated from planktonic bacteria to provide at least one washed biofilm coated bead substantially free of planktonic bacteria. The washed biofilm coated bead is then aseptically transferred to a vessel such as a well of a well plate. A growth medium, a metabolic dye and a known amount of the active agent of interest are then added to the or each vessel containing a washed biofilm coated bead. The growth medium, metabolic dye, active agent of interest and biofilm coated bead are then incubated in the or each vessel. Any change in color intensity of the metabolic dye in the or each vessel is measured and the activity of the active agent against the bacterial biofilm is determined based on the color intensity in the or each vessel.

The active agent can be any antibacterial or potential antibacterial moiety, such as an antibiotic, phage, biocide, disinfectant or heavy metal.

In some embodiments, the active agent is an antibiotic. Thus, the methods disclosed herein provide a quick, accurate and reliable method to determine minimum biofilm inhibitory concentration of antibiotic (MBIC) to biofilm infection. Any known antibiotic can be assayed using the methods disclosed herein. Some common antibiotics include those that target the bacterial cell wall (penicillins and cephalosporins) or the cell membrane (polymyxins), those that interfere with essential bacterial enzymes (rifamycins, lipiarmycins, quinolones, and sulfonamides), protein synthesis inhibitors (macrolides, lincosamides, and tetracyclines), gram-negative antibiotics, gram-positive antibiotics, broad-spectrum antibiotics, cyclic lipopeptides (such as daptomycin), glycylcyclines (such as tigecycline), oxazolidinones (such as linezolid), and lipiarmycins (such as fidaxomicin).

In some other embodiments, the active agent is a phage. Thus, the methods disclosed herein provide a quick, accurate and reliable method of matching phage to biofilms. Examples of such phage include, but is not limited to, a phage selected from APT-PJI-01, APT-PJI-02, APT-PJI-03, APT-PJI-04, APT-PJI-05, APT-PJI-06, APT-PJI-07, APT-PJI-08, APT-PJI-09, APT-PJI-10, APT-PJI-11, APT-PJI-12, APT-PJI-13, APT-PJI-14, APT-PJI-15, APT-PJI-16, APT-PJI-17, APT-PJI-18, APT-PJI-20, APT-PJI-21, APT-PJI-22, APT-PJI-23, APT-PJI-24, APT-PJI-25, APT-PJI-26, APT-PJI-27, APT-PJI-28, APT-PJI-29, APT-PJI-30, APT-PJI-31, APT-PJI-32, APT-PJI-33, APT-PJI-34, APT-PJI-35, APT-PJI-36, APT-PJI-37, APT-PJI-38, APT-PJI-39, APT-PJI-40, APT-PJI-41, APT-PJI-42, APT-PJI-43, APT-PJI-44, APT-PJI-45, APT-PJI-46, APT-PJI-47, APT-PJI-48, APT-PJI-49, and/or APT-PJI-50 as disclosed in PCT/US21/25794, which is herein incorporated by reference in its entirety.

In some other embodiments, the active agent comprises an antibiotic and a bacteriophage. Thus, the methods disclosed herein provide quick, accurate and reliable phage-antibiotic synergy testing to biofilm infection.

The active agent may also be an antibiotic-antibiotic combination, antibiotic-antibacterial compound combination, phage-phage combination, or phage-antibacterial compound combination and the methods disclosed herein can be used to test synergy of any such combination to biofilm infection.

The MBIC of an active agent can be determined by adding a first known amount of the active agent of interest to a first well containing a washed biofilm coated bead and adding at least one other known concentration of the active agent of interest to at least one other well containing a washed biofilm coated bead. The activity of the active agent at different concentration on the bacterial biofilm can be determined based on differences between the intensity of the color in the first well and the other well(s). Thus also disclosed herein is a method for determining the minimum biofilm inhibitory concentration of an active agent. The method comprises culturing a bacterial colony in a culture medium in contact with at least one bead under conditions to form at least one biofilm coated bead. The at least one biofilm coated bead is separated from planktonic bacteria to provide at least one washed biofilm coated bead substantially free of planktonic bacteria which is then transferred to a well of a multi-well plate. A growth medium, a biofilm dye and a first known amount of an active agent of interest is added to a first well containing a washed biofilm coated bead and a growth medium, a biofilm dye and a second known amount of the active agent of interest is added to a second well containing a washed biofilm coated bead. The growth medium, biofilm dye, active agent of interest and biofilm coated bead in the first and second wells are incubated and any change in color intensity of the metabolic dye in the first and second wells is measured. The minimum biofilm inhibitory concentration of the active agent on the bacterial biofilm can then be determined based on differences between the intensity of the color in the first well and the second well.

The beads can be made from glass, metal, rubber, plastic, composite, etc and have a surface suitable for biofilm growth. For example, beads may be coated with a suitable material such as hydroxyapatite, polystyrene, rubber latex, polycarbonate, polyvinyl alcohol, dextran, silica, carbon, or other suitable material. The beads can be any suitable size, such as, for example, at least 2 mm to at least 8 mm in size, or at least 2 mm to at least 4 mm in size, or at least 2 mm to at least 6 mm in size, or at least 2 mm to at least 8 mm in size, or 4 mm to at least 8 mm in size, or at least 4 mm to at least 6 mm in size, or at least 6 mm to at least 8 mm in size, or at least 2 mm in size, or at least 4 mm in size, or at least 6 mm in size, or at least 8 mm in size. In certain embodiments, the beads are 4 mm beads.

Optionally, the beads may be magnetic. The beads are sterilized prior to use using any known sterilization methods.

A bacterial colony is cultured in a culture medium in contact with the beads under conditions to form biofilm coated beads. The bacterial colony may be a single bacterial colony of interest. The bacteria may be gram-positive, such as Bacillus spp, Listeria monocytogenes, Staphylococcus spp, and lactic acid bacteria, including Lactobacillus plantarum and Lactococcus lactis or it may be a gram-negative species such as Escherichia coli, or Pseudomonas aeruginosa. Preferred examples of Staphylococcus, include, but are not limited to S. aureus, S. epidermidis, S. capitis, S. caprae, S. warneri, or S. lugdunensis. In further preferred embodiments, the bacterium isolate may also have bacterium selected from at least one of Enterococcus spp., including E. fecalis and Enterococcus fecium, other coagulase-negative Staphylococcus species, including S. simulans, Streptococcus spp., including Lancefield groups A, B, C and G, S. agalactiae and S. pneumoniae, aerobic gram-negative bacteria including E. coli, other Enterobacteriaceae including Enterobacter clocae, Clostridium spp., Actinomyces spp., Peptostreptococcus spp., Cutibacterium acnes, Klebsiella pneumoniae, P. aeruginosa and Bacteroides fragilis. In even further embodiments, the bacteria can be a multi-drug resistant (“MDR”) bacteria. Examples of MDR bacteria that may be included in the screen include, but are not limited to the “ESKAPE” pathogens (Enterococcus faecium, Staphylococcus aureus, Klebsiella pneumonia, Acinetobacter baumannii, Pseudomonas aeruginosa, and Enterobacter sp), which are often nosocomial in nature and can cause severe local and systemic infections. Specifically, these include, e.g., methicillin-resistant Staphylococcus aureus (MRSA); vancomycin-resistant Enterococcus faecium (VRE); carbapenem-resistant Klebsiella pneumonia (NDM-1); MDR-Pseudomonas aeruginosa; and MDR-Acinetobacter baumannii.

Any suitable culture medium can be used to culture the bacterial colony. In some embodiments, the culture medium is tryptic soy broth (TSB). The culture medium may be inoculated with the single bacterial colony from fresh streaked plate as starter culture. The bacterial colony may be cultured in any suitable vessel in the presence of the beads, such as a well plate containing approximately 20 beads per well.

The bacterial colony may be cultured by incubation without shaking at 37° C. for a suitable period of time, such as 24 hours. The bacteria will grow in the media and form the biofilm on the surface of the beads under these conditions. This method provides a relatively simple way to establish biofilm.

The biofilm coated beads are separated from planktonic bacteria to provide washed biofilm coated beads. Advantageously, the methods disclosed herein provide a simple, effective and reliable method for removing planktonic bacteria from the biofilm on the beads and, thereby preventing, minimizing or reducing interference from planktonic bacteria on the assay. Planktonic bacteria are free floating and loosely attached bacteria to the beads. They can be removed with a serological pipette to leave the beads in the well. The beads can then be removed with a suitable solution, such as PBS. The solution can then be removed. The bead can be washed again for as many cycles as desired. This process will remove substantially all planktonic bacterial cells from the beads. Suspension of the beads in the wells during washing cycles minimizes biofilm disruption.

The washed biofilm coated beads are then transferred to a suitable vessel, such as a well of a well plate so that there is one bead in each vessel. The washed biofilm coated beads can be transferred using any suitable method, including with sterilized forceps, sterilized pipettes or using a magnetic if the beads are magnetic. If desired, the washed biofilm coated beads can be transferred aseptically.

In some embodiments, the assay may be used to assess biofilm formation in patients with implanted devices or prosthetics, wherein bacteria are isolated from the patient(s) and bead material is chosen that matches the material of the implanted device in the patient(s). In other embodiments, the bacteria is isolated from a patient suffering from an infection, wherein the infection is selected from: a prosthetic joint infection (PJI), a chronic bacterial infection, an acute bacterial infection, a refractory infection, an infection associated with a biofilm, an infection associated with an implantable device, diabetic foot osteomyelitis (DFO), diabetic foot infection (DFI), a lung infection, such as those occurring in patients having cystic fibrosis (CF) or pneumonia, an urinary tract infection (UTI), a skin infection, such as acne or atopic dermatitis, an eye infection, such as conjunctivitis, bacterial kaeratitis, endophthalmitis, or blepharitis, sepsis or other blood infection.

The well plate can be any suitable well plate, such as a 96 well HRQT Biolog plate. As used herein, “HRQT” stands for “Host Range Quick Test.” The HRQT assay can be run on a Biolog machine which can measure the change in the tetrazolium dye color because of the bacterial metabolic activities. Currently we are using this test for matching of phage to control PLANKTONIC (free floating) bacterial population.

A growth medium, a biofilm dye and a known amount of the active agent of interest are then added to each well.

The growth medium can be any suitable growth medium such as tryptic soy broth (TSB).

The biofilm dye can be any known dye such as safranin, metabolic dyes such as tetrazolium-based dyes, rezazurin dye, and other fluorescent labels. In certain specific embodiments, the biofilm dye is a metabolic dye, more particularly a tetrazolium-based dye such as 3-(4,5-dimethylthiazol-2-yl)-2,5-diphenyltetrazolium bromide (MTT).

The biofilm dye can be added as a solution in the growth medium, such as a 1% solution of biofilm dye in growth medium.

The growth medium, biofilm dye, active agent of interest and biofilm coated bead are then incubated in the wells. Advantageously, the 96 well HRQT/Biolog plate with wells containing the growth medium, biofilm dye, active agent of interest and biofilm coated bead are incubated in the HRQT/Biolog machine which will take reading color readings after every 15 minutes.

Any change in color intensity of the biofilm dye in each well is measured in the HRQT/Biolog machine. The camera of HRQT/Biolog machine will take pictures from the top of the plate to measure the change in the color of biofilm dye. As the dye will change in color or color intensity as the bacteria metabolize in the presence of the active agent the activity of the active agent against the bacterial biofilm can be determined based on the color or the color intensity in each well. The automation of the HRQT can provide the results in real time.

Alternatively, the activity of the active agent against the bacterial biofilm could be measured and determined using an optical density-based plate reader

The methods disclosed herein can be used to determine a minimum inhibitory concentration (MIC), a minimum biocidal concentration (MBC) or a minimum biofilm eradication concentration (MBEC) of an active agent of interest and can be used in diagnostic labs, hospitals or research labs.

### EXAMPLES

### Example 1—Method to Determine Minimum Biofilm Inhibitory Concentration of Antibiotic (MBIC) to Biofilm Infection

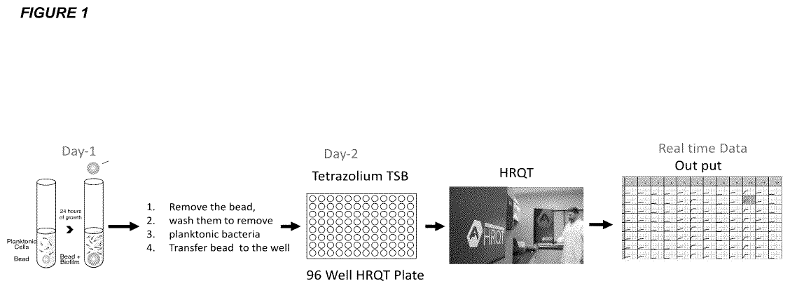

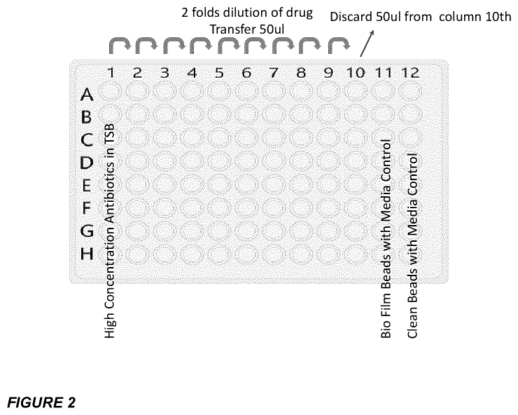

The MBIC of an antibiotic was determined by the following method:
Add 4 mm sterilized beads in a 6 well plate, approximately 20 beads per well.
Add 5 mL TSB media to each well of the 6 well plate and inoculate a single bacterial colony from a fresh streaked plate as starter culture.
Incubate the plate without shaking at 37° C. for 24 hours. The bacteria will grow in the media and form the biofilm on the surface of the glass beads as shown in step 1 of FIG. 1.
Remove the planktonic culture (free floating and loosely attached bacteria to the beads) with a serological pipette and leave the beads in the well.
Add 7 mL PBS to wash the beads, remove the PBS and wash it again with PBS. This will remove all planktonic bacterial cells from the beads.
Use sterilized forceps to remove the beads and transfer them to a HRQT Biolog plate so that there is one bead per well. In the 12^{th} column add biofilm free beads as control. The layout of the 96 well plate with beads is shown in FIG. 2.
Add 100 ul of TSB media comprising 1% metabolic dye tetrazolium to each well.
In the first column add an antibiotic of interest with highest concentration made in tetrazolium TSB medium.
Using a multi-pipette mix the drug and transfer 50 μl to next column, change the micropipette tips with new tips, mix the drug in the second column, pick 50 μl of the drug-media mixture and transfer to the next until the 10^{th} column of the Biolog plate is reached.
Do not add drug in the 11th column as this is the drug free control.
Incubate the plate in the HRQT/Biolog machine and take a reading after every 15 minutes.
The tetrazolium metabolic dye will change color intensity as the bacteria metabolize it in the presence of phage.
The camera of HRQT/Biolog machine will take the pictures from the top to measure the change in the color of metabolic dye. The presence of a bead in the well will not hinder the functioning of machine.

This assay can reduce the time for measuring MBIC to biofilm testing to −1.5 days as compared to the conventional methods and it can provide the results in real time. The assay is controlled, efficient and produces robust and reproducible results.

Furthermore, there is no MBIC method for commercial use in the hospital. Currently, a physician uses a MIC value measured from the planktonic cells to guess the MBIC. In contrast, the present method can be used as commercial diagnostic test for MBIC for bacterial biofilm.

### Example 2—Method of Matching Phage for Anfibiofilm Activity by Using Metabolic Dye and Spherical Beads

The antibiofilm activity of phage can be determined by the following method:
Add 4 mm sterilized beads in 6 well plate, approximately 20 beads per well.
Add 5 mL TSB media in each well of 6 well plate and inoculate single bacterial colony from fresh streaked plate as starter culture.
Incubate the plate without shaking at 37° C. for 24 hours. The bacteria will grow in the media and form the biofilm on the surface of the glass beads as shown in step 1 of FIG. 1.
Remove the planktonic culture (free floating and loosely attached bacteria to the beads) with a serological pipette and leave the beads in the well.
Add 7 mL PBS to wash the beads, remove the PBS and wash it again with PBS. It will remove all planktonic bacterial cells from the beads.
Using sterilized forceps, remove the beads and transfer to a HRQT Biolog plate so that there is one bead per well.
Add 100 ul of TSB media supplemented with 1% tetrazolium metabolic dye and 10⁷ PFU/mL phage of interest to each well. In the phage free control, do not add phage.
Incubate the plate in a HRQT (OmniLog) machine and take a reading after every 15 minutes.
The tetrazolium metabolic dye will change its color intensity as the bacteria metabolize and proliferate in the presence of phage.
The camera of HRQT/Biolog machine will take the pictures from the top and the software will measure the change in the color intensity of the metabolic dye over time. The presence of bead in the well will not hinder the functioning of machine.

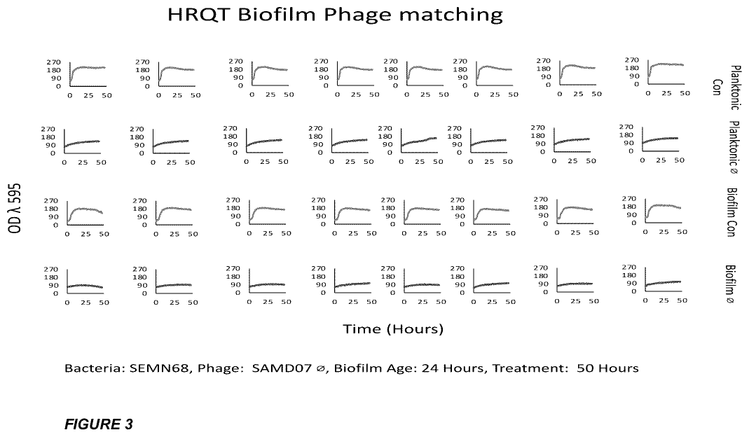

The above method was carried out using SEMN68 bacteria, SAMD07 phage (1) using biofilm that had been aged for 24 hours and with a treatment time of 50 hours. The results are shown in FIG. 3 and they show that biofilm bacteria metabolic activity is less in the presence of phage than the phage free control.

### Example 3—Method of Phage Antibiotic Synergy Testing by Using Bacterial Metabolic Dye and Spherical Beads

Any synergy between a phage of interest and an antibiotic of interest can be determined by the following method:
Add 4 mm sterilized beads in 6 well plate, approximately 20 beads per well.
Add 5 mL TSB media in each well of the 6 well plate and inoculate single bacterial colony from fresh streaked plate as starter culture.
Incubate the plate without shaking at 37° C. for 24 hours. The bacteria will grow in the media and form the biofilm on the surface of the glass beads as shown in step 1 of FIG. 1.
Remove the planktonic culture (free floating and loosely attached bacteria to the beads) with a serological pipette and leave the beads in the well.
Add 7 mL PBS to wash the beads, remove the PBS and wash it again with PBS. This will remove all planktonic bacterial cells from the beads.
Using sterilized forceps, remove the beads and transfer to a 96 well HRQT Biolog plate so that there is one bead per well.
Add 100 μl of TSB media comprising 1% metabolic dye tetrazolium and 10⁷ PFU/mL phage with different combinations of antibiotics of interest in each well. In the phage free control, do not add phage.
Incubate the plate in a HRQT/Biolog machine (OmniLog) and take a reading after every 15 minutes.
The tetrazolium metabolic dye will change in color intensity as the bacteria metabolizes and grows in the presence of phage.
The camera of HRQT/Biolog machine will take the pictures from the top to measure the change in the color of metabolic dye. The presence of bead in the well will not hinder the functioning of machine.

To detect dormant cells, after the completion of treatment, remove the beads from the 96 well plate, wash them with PBS and place them in new 96 well plate along with fresh TSB media.

If the beads have viable cells which were dormant in the presence of antibiotic or phage, they will start growing in the fresh TSB media and change the intensity of the color of metabolic dye.

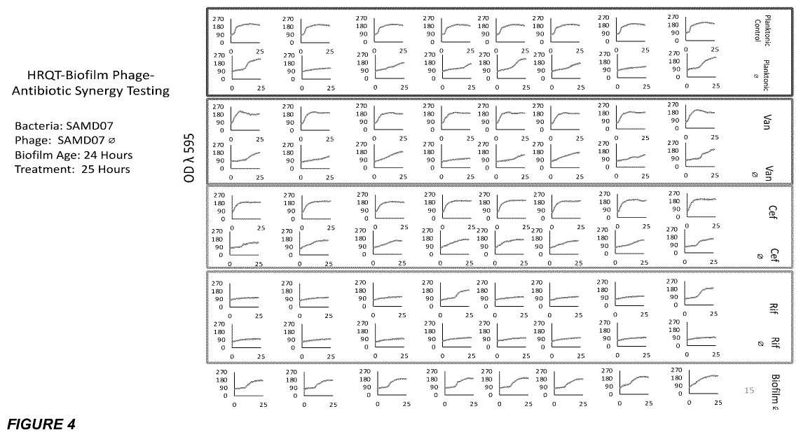

The results are shown in FIG. 4, where the uppermost panel shows the metabolic activity of planktonic bacteria in the presence of phage and absence of phage (Control), the second uppermost panel shows the metabolic activity of biofilm bacteria in the presence of 1× minimum inhibitory concentration (MIC) of Vancomycin antibiotic and 1× MIC vancomycin and phage, the third uppermost panel shows the metabolic activity of biofilm bacteria in the presence of 1× minimum inhibitory concentration (MIC) of Ceftriaxone antibiotic and 1× MIC Ceftriaxone and phage, the fourth uppermost panel shows the metabolic activity of biofilm bacteria in the presence of 1× minimum inhibitory concentration (MIC) of Rifampicin antibiotic and 1× MIC Rifampicin and phage, and the bottom panel shows the metabolic the metabolic activity of biofilm bacteria in the presence of phage only.

The results provided herein demonstrate that these assays can be a quick and efficient method for biofilm-phage matching since it reduces the time for screening phage libraries for anti-biofilm testing to ˜1.5 days as compare to the conventional methods which takes 3-5 days to produce the results. The assays provide the results in real time and the results are robust and reproducible.

The assays can be used as commercial diagnostic test for phage matching for bacterial biofilm and can help screen a large collection of phages against biofilm of various bacterial targets to find biofilm specialized phages. This same assay can also be used to measure synergy between multiple phage, between phage and antibiotics, or between antibiotics.

The reference to any prior art in this specification is not, and should not be taken as, an acknowledgement or any form of suggestion that such prior art forms part of the common general knowledge.

It will be understood that the terms “comprise” and “include” and any of their derivatives (e.g. comprises, comprising, includes, including) as used in this specification, and the claims that follow, is to be taken to be inclusive of features to which the term refers, and is not meant to exclude the presence of any additional features unless otherwise stated or implied.

In some cases, a single embodiment may, for succinctness and/or to assist in understanding the scope of the disclosure, combine multiple features. It is to be understood that in such a case, these multiple features may be provided separately (in separate embodiments), or in any other suitable combination. Alternatively, where separate features are described in separate embodiments, these separate features may be combined into a single embodiment unless otherwise stated or implied. This also applies to the claims which can be recombined in any combination. That is a claim may be amended to include a feature defined in any other claim. Further a phrase referring to “at least one of” a list of items refers to any combination of those items, including single members. As an example, “at least one of: a, b, or c” is intended to cover: a, b, c, a-b, a-c, b-c, and a-b-c.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

Please note that the following claims are provisional claims only, and are provided as examples of possible claims and are not intended to limit the scope of what may be claimed in any future patent applications based on the present application. Integers may be added to or omitted from the example claims at a later date so as to further define or re-define the scope.

### REFERENCES

Chaudhry W N, Concepcion-Acevedo J, Park T, Andleeb S, Bull J J, Levin B R. Synergy and Order Effects of Antibiotics and Phages in Killing Pseudomonas aeruginosa Biofilms. PLoS One. 2017 Jan. 11; 12(1):e0168615. doi: 10.1371/journal.pone.0168615. PMID: 28076361; PMCID: PMC5226664.
Comeau A M, Tetart F, Trojet S N, Prere M F, Krisch H M (2007) Phage-antibiotic synergy (PAS): β-lactam and quinolone antibiotics stimulate virulent phage growth. PLoS One 2:e799
Merritt, J. H., Kadouri, D. E., & O'Toole, G. A. (2005). Growing and analyzing static biofilms. Current protocols in microbiology, Chapter 1, Unit-1B.1. https://doi.org/10.1002/9780471729259.mc01b01s00.
Nair S, Desai S, Poonacha N, Vipra A, Sharma U. Antibiofilm Activity and Synergistic Inhibition of Staphylococcus aureus Biofilms by Bactericidal Protein P128 in Combination with Antibiotics. Antimicrob Agents Chemother. 2016 Nov. 21; 60(12):7280-7289. doi: 10.1128/AAC.01118-16. PMID: 27671070; PMCID: PMC5119008.

1. A method for determining the anti-biofilm activity of an active agent on a bacterial biofilm, the method comprising:
culturing a bacterial colony in a culture medium in contact with at least one bead under conditions to form at least one biofilm coated bead;
separating the at least one biofilm coated bead from planktonic bacteria to provide at least one washed biofilm coated bead substantially free of planktonic bacteria;
transferring a washed biofilm coated bead to a vessel;
adding a growth medium, a biofilm dye and a known amount of an active agent of interest to the vessel containing a washed biofilm coated bead;
incubating the growth medium, biofilm dye, active agent of interest and biofilm coated bead in the or each vessel;
measuring any change in color intensity of the metabolic dye in the or each vessel; and
determining the activity of the active agent on the bacterial biofilm based on the color intensity in the or each well.

2. The method of claim 1, wherein the active agent is an antibiotic.

3. The method of claim 1, wherein the active agent is a bacteriophage.

4. The method of claim 1, wherein the active agent comprises an antibiotic-antibiotic combination, antibiotic-antibacterial compound combination, a phage-phage combination, or phage-antibacterial compound combination.

5. The method of claim 2, comprising:
adding a first known amount of the active agent of interest to a first vessel containing a washed biofilm coated bead;
adding at least one other known concentration of the active agent of interest to at least one other vessel containing a washed biofilm coated bead; and
determining the activity of the active agent on the bacterial biofilm based on differences between the intensity of the color in the first well and the other vessel(s).

6. The method according to claim 1, wherein the biofilm dye is a metabolic dye.

7. The method of claim 2, wherein the metabolic dye is 3-(4,5-dimethylthiazol-2-yl)-2,5-diphenyltetrazolium bromide (MTT).

8. The method of claim 2, wherein absorbance is measured at 400-700 nanometers.

9. The method according to claim 1, wherein the growth medium is tryptic soy broth (TSB).

10. The method according to claim 1, wherein the bacterial colony is a single bacterial colony.

11. The method according to claim 1, wherein the bead used is magnetic.

12. The method according to claim 1, wherein the bacterial colony is isolated from a patient with an implanted device and/or prosthetic, alu and the bead material matches that of the implanted device and/or prosthetic.

13. A method for determining the minimum biofilm inhibitory concentration of an active agent, the method comprising:
culturing a bacterial colony in a culture medium in contact with at least one bead under conditions to form at least one biofilm coated bead;
separating the at least one biofilm coated bead from planktonic bacteria to provide at least one washed biofilm coated bead substantially free of planktonic bacteria;
transferring a washed biofilm coated bead to a well of a multi-well plate;
adding a growth medium, a biofilm dye and a first known amount of an active agent of interest to a first well containing a washed biofilm coated bead;
adding a growth medium, a biofilm dye and a second known amount of the active agent of interest to a second well containing a washed biofilm coated bead; and
incubating the growth medium, biofilm dye, active agent of interest and biofilm coated bead in the first and second wells;
measuring any change in color intensity of the metabolic dye in the first and second wells; and
determining the minimum biofilm inhibitory concentration of the active agent on the bacterial biofilm based on differences between the intensity of the color in the first well and the second well.

14. The method of claim 1, wherein the bacteria colony is isolated from a patient suffering from an infection, wherein the infection is selected from: a prosthetic joint infection (PJI), a chronic bacterial infection, an acute bacterial infection, a refractory infection, an infection associated with a biofilm, an infection associated with an implantable device, diabetic foot osteomyelitis (DFO), diabetic foot infection (DFI), a lung infection, such as those occurring in patients having cystic fibrosis (CF) or pneumonia, an urinary tract infection (UTI), a skin infection, such as acne or atopic dermatitis, an eye infection, such as conjunctivitis, bacterial kaeratitis, endophthalmitis, or blepharitis, sepsis or other blood infection.
